(19) United States
(12) Patent Application Publication
(10) Pub. No.: US 2010/0003348 A1
(43) Pub. Date: Jan. 7, 2010

(54) USE OF CLAYS FOR TREATING COELIAC DISEASE

(75) Inventor: Héléne Mathiex-Fortunet, Paris (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: IPSEN PHARMA S.A.S., Boulogne-Billancourt (FR)

(21) Appl. No.: 12/525,886

(22) PCT Filed: Feb. 6, 2008
(86) PCT No.: PCT/FR08/00142

§ 371 Date: Aug. 5, 2009

(30) Foreign Application Priority Data
Feb. 6, 2007 (FR) 0700821

(51) Int. Cl. A61K 33/12; A61K 33/06; A61P 1/00

(52) U.S. Cl. 424/683; 424/684

(57) ABSTRACT

The invention relates to the use of a clay for the preparation of a medicament for treating coeliac disease. Preferably, the clay used is of smectite type.

The present Application relates to the use of a clay for preparing a medicament intended to treat or prevent coeliac disease.

Coeliac disease is an auto-immune disease; it is characterized by a food intolerance to certain gluten components such as prolamines. The latter are present in significant quantities in numerous cereals, in particular wheat, rye and barley. Coeliac disease is the consequence of a digestive hypersensitivity to these gluten components. In certain genetically predisposed subjects, the ingestion of this protein will trigger an exaggerated immune reaction which leads to lesions of the intestinal mucous membrane. Apart from genetic factors, other factors (infectious, viral and/or bacterial) could be involved.

The prevalence of this disease varies greatly from one country to another for reasons which are still poorly determined; for example, it is 1/100 in Europe (1/300 to 1/125 in Ireland) and 1/300 in the United States. It affects girls more than boys. The first signs of the disease appear very early in life, generally between the ages of six months and two years, but they can be detected much later, sometimes even in adulthood.

At present, its treatment is based solely on the exclusion of gluten from the diet, which in the majority of cases, ensures a clinical cure and prevents complications such as lymphoma or osseous demineralization. Even if the gluten-free diet appears simple in principle, it is very restrictive, socially incapacitating and very often difficult to implement without medical support.

A subject of the present invention is therefore the use of a clay for preparing a medicament intended to treat or prevent coeliac disease.

A more particular subject of the present invention is the use of a clay for preparing a medicament intended to prevent coeliac disease.

A more particular subject of the present invention is also the use of a clay for preparing a medicament intended to treat coeliac disease.

Clay is a known natural product which nowadays is used both in cosmetic applications and in the medical field for treating certain pathologies such as diarrhoea.

The clay used according to the invention can be a clay of the smectite family. Smectites represent a particular family of clay in which dioctahedral species such as montmorillonite and beidellite, and trioctahedral species such as hectorite and saponite are found.

The clay used according to the present invention is preferably a smectite, and very preferably a dioctahedral smectite. Preferably, the dioctahedral smectite is a montmorillonite or a beidellite or an intermediate crystallographic structure between the two crystal-chemical poles: montmorillonite and beidellite. This intermediate crystallographic structure can be close to the montmorillonite pole and even very close to the montmorillonite pole; it can also be close to the beidellite pole and even very close to the beidellite pole.

Preferably, a dioctahedral smectite according to the invention is a montmorillonite or an intermediate structure close to the montmorillonite pole, and very preferably a montmorillonite or an intermediate structure very close to the montmorillonite pole.

Also very preferentially, the clay used is the smectite called “diosmectite” and marketed under the trademark Smecta®.

The pharmaceutical compositions comprising a clay can be in the form of solids, for example powders, granules, tablets or gelatin capsules. The appropriate solid supports can be, for example, talc, sugars, lactose, dextrin, gelatin, cellulose and its esters.

The pharmaceutical compositions comprising a clay can also be presented in liquid form, for example, solutions, suspensions or syrups. Appropriate liquid supports can be, for example, water, organic solvents such as alcohols (glycerol, glycols), as well as their mixtures, in varying proportions, in water. The composition will also comprise preservatives such as benzoic acid esters.

In the above pharmaceutical compositions. the clay can also be combined with colourings and/or flavourings.

The administration of the clay according to the invention can be carried out by oral or enteral route. Preferably, the clay and in particular the smectites as defined above are administered by oral route.

The daily administration dose of clay is the usual dose recommended for this product. In the particular case of the smectite called “diosmectite”, it can be administered in a maximum daily dose of 18 g/day.

Unless otherwise specified, all the technical and scientific terms used here have the same meaning as that usually understood by an ordinary specialist in the field to which this invention belongs.

Pharmacological Part

For individuals suffering from coeliac disease, the diagnosis is made by oeso-gastroduodenal endoscopy and duodenal biopsies, the histological analysis of which reveals a more or less pronounced villositary atrophy. Thus, the effectiveness of a clay in the treatment of coeliac disease can be monitored by histology of the intestinal mucous membrane.

The activity of a clay of smectite type such as the smectite called “diosmectite” (Smecta®) in the treatment of coeliac disease can be evaluated according to the following experimental protocol:

The first clinical study can be a proof of efficacy study (phase II). The smectite could be administered in a dose of 3 g three times per day for at least 6 months.

During the study, the clinical state is monitored, in particular the digestive symptoms including diarrhoea and abdominal flatulence, the nutritional, in particular biological, state, tests for absorption of macro- and micro-nutrients, the immunological state, in particular the titre of antibodies which are markers of coeliac disease, the state of the intestinal mucous membrane, in particular the improvement in the villositary atrophy and inflammation.

The development plan then comprises a randomized, double blind, placebo-controlled Phase III study of patients who have recently been diagnosed and have started on the gluten-free diet. The efficacy is evaluated by the time taken for normalization of the intestinal mucous membrane, and by the same parameters as those monitored in Phase II.

1-9. (canceled)

10. A method for treating or preventing coeliac disease comprising administering a pharmaceutical composition comprising a therapeutically effective amount of a clay.

11. The method of claim 1, wherein the coeliac disease is prevented.

12. The method of claim 1, wherein the coeliac disease is treated.

13. The method of claim 1, wherein the clay is a smectite.

14. The method of claim 13, wherein the smectite is dioctahedral smectite.

15. The method of claim 14, wherein the dioctahedral smectite has a montmorillonite or a beidellite or an intermediate crystallographic structure between the montmorillonite crystal-chemical pole and the beidellite crystal-chemical pole.

16. The method of claim 15, wherein the dioctahedral smectite has a montmorillonite crystallographic structure.

17. The method of claim 15, wherein the dioctahedral smectite has an intermediate crystallographic structure closer to the montmorillonite crystal-chemical pole.

18 The method of claim 15, wherein the dioctahedral smectite has an intermediate crystallographic structure very close to the montmorillonite crystal-chemical pole.

19. The method of claim 15, wherein the dioctahedral smectite has a beidellite crystallographic structure.

20. The method of claim 15, wherein the dioctahedral smectite has an intermediate crystallographic structure closer to the beidellite crystal-chemical pole.

21. The method of claim 15, wherein the dioctahedral smectite has an intermediate crystallographic structure very close to the beidellite crystal-chemical pole.

22. The method of claim 1, wherein the clay is diosmectite.

23. The method of claim 1, wherein the pharmaceutical composition comprises a solid support.

24. The method of claim 23, wherein the solid support comprises talc, sugars, lactose, dextrin, gelatin, cellulose, cellulose esters or mixtures thereof.

25. The method of claim 1, wherein the pharmaceutical composition comprises a liquid support.

26. The method of claim 25, wherein the solid support comprises water, organic solvents or mixtures thereof.

27. The method of claim 26, wherein the organic solvent comprises glycerols, glycols or mixtures thereof.

28. The method of claim 1, wherein the pharmaceutical composition is administered orally or enterally.

29. A method for treating or preventing coeliac disease comprising administering a maximum daily dose of 18 g/day of a clay.
